(19) United States
(12) Patent Application Publication
(10) Pub. No.: US 2006/0204486 A1
(43) Pub. Date: Sep. 14, 2006

(54) Pharmaceutical compositions for the treatment and/or prevention of schizophrenia and related diseases

(75) Inventors: Robert Pyke, New Fairfield, CT (US); Angelo Ceci, Mittlebiberach (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 11/364,306

(22) Filed: Feb. 28, 2006

Related U.S. Application Data
(60) Provisional application No. 60/658,566, filed on Mar. 4, 2005.

(51) Int. Cl. A61K 38/54; A61K 31/551; A61K 31/5415; A61K 31/519; A61K 31/496; A61K 31/445; A61K 31/405; A61K 31/198

(52) U.S. Cl. 424/94.2; 514/254.06; 514/220; 514/259.41; 514/419; 514/317; 514/561; 514/225.8

(57) ABSTRACT

The invention relates to new pharmaceutical compositions for the treatment and/or prevention of schizophrenia and methods for the preparation thereof. In a preferred embodiment, the instant invention is directed to pharmaceutical combinations comprising flibanserin as one active ingredient in combination with at least one additional active ingredient for the treatment and/or prevention of schizophrenia and methods for the preparation thereof.

The invention relates to new pharmaceutical compositions for the treatment and/or prevention of schizophrenia and related diseases and methods for the preparation thereof. In a preferred embodiment, the instant invention is directed to pharmaceutical combinations comprising flibanserin as one active ingredient in combination with at least one additional active ingredient for the treatment and/or prevention of schizophrenia and related diseases and methods for the preparation thereof.

### BACKGROUND OF THE INVENTION

The invention relates to new pharmaceutical compositions for the treatment and/or prevention of schizophrenia and related diseases and methods for the preparation thereof. In one embodiment, the instant invention is directed to pharmaceutical combinations comprising a therapeutically effective amount of flibanserin 1 as one active ingredient of combination with a therapeutically effective amount of at one or more, preferably one additional antipsychotic drug 2 for the treatment and/or prevention of schizophrenia and related diseases and methods for the preparation thereof.

The compound 1—[2—(4—(3—trifluoromethyl-phenyl)ethyl]-2,3-dihydro-1H-benzimidazol-2-one (flibanserin) is disclosed in form of its hydrochloride in European Patent Application EP-A-526434 and has the following chemical structure:

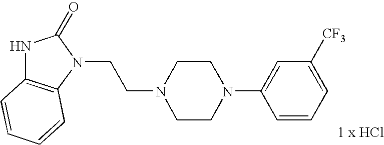

Flibanserin shows affinity for the 5-HT_{1A},5-HT₂₋ and D₄-receptor. It is therefore a promising thereapeutic agent for the treatment of a variety of diseases, for instance depression, schizophrenia, Parkinson, anxiety, sleep disturbances, sexual and mental disorders and age associated memory impairment.

One embodiment of the invention is directed to pharmaceutical compositions comparing a therapeutically effective amount of flibanserin 1 in combination with a therapeutically effective amount of one or more additional antipsychotic drugs 2.

Another embodiment of the invention is directed to pharmaceutical compositions comprising a therapeutically effective amount of flibanserin 1 in composition with a therapeutically effective amount of one or more, preferably one antipsychotic drug 2 selected from the group consisting of 5-HT_{1A} agnostics, dopamine modulators, sodium channel blockers, 5-HT uptake inhibitors, D3 antagonists, D2 antagonists, D1 antagonists, D1 agonists, secretin agonist, phospholipase A2 inhibitors, 5-HT2 antagonists, 5HT6 antagonists, COX 2 inhibitors, 5-HT_{2A} antagonists, 5HT_{2C} modulators, NK3 antagonists, alpha 1 adrenoreceptor antagonists, alpha 2 adrenoreceptor antagonists, AMPA modulators and NK 3 antagonists.

Especially preferred are pharmaceutical compositions comprising a therapeutically effective amount of flibanserin 1 in combination with a therapeutically effective amount of one or more, preferably one antipsychotic drug 2 selected from the group consisting of D2 antagonists.

The compositions according to the invention may contain flibanserin 1 and the one or more additional antipsychotic drugs 2 in a single formulation or in separate formulations. If flibanserin and the one or more additional antipsychotic drugs are present in separate formulations these separate formulations may be administered simultaneously or sequentially.

A preferred embodiment according to the invention is directed to pharmaceutical compositions comprising a therapeutically effective amount of flibanserin 1 and a therapeutically effective amount of one or more, preferably one additional antipsychotic drug 2, optionally in combination with a pharmaceutically acceptable excipient.

Examples of suitable additional antipsychotic drugs include Chlorpromazine. Thioridazine, Haloperidol, Perphenazine, Thiothixene, Trifluoperazine, Fluphenazine, Clozapine, Risperidone, Olanzapine, Quetiapine, Pimozide, Aripiprazole, Ziprasidone, Perospirone, Nemonapride, Sertindole, Levosulpiride, Tandospirone, Bifeprunox, Asenapine, Paliperidone, Mifepristone, Lamotrigine, Iloperidone, Blonanserin, DU-125530, Lurasidone, ACP-103, Idazoxan, Org-24448, CX-516, Aplindore, SLV-313, SLV-310, Ocaperidone, PNU-170413, POL-255, ABT-089 Talnetant, NE-100, LAX-101, LAX-111, RG-1068 (Secretin), Dexefraoxan, Dihydrexidine, SM-13496, D-Serine, Osanetant, EMR-62218, SB-399885, TC-1698, SR-147778, SLV-319, SSR-181507, AVE-5997, PNU-177864, Abaperidone, SSR-146977, Neboglamine, Lamictal XT, N-Desmethylclozapine, Topiramate and cycloserine, optionally in form of the pharmaceutically acceptable acid addition salts, in form of the hydrates and/or solvates and optionally in the form of the individual optical isomers, mixtures of the individual enantiomers or racemates thereof.

Flibansering 1 may be used in form of the free base, optionally in form of its pharmaceutically acceptable acid addition salts and/or optionally in form of the hydrates and/or solvates thereof. Suitable acid addition salts include for example those of the acids selected from, succinic acid, hydrobromic acid, acetic acid, fumaric acid, maleic acid, methanesulphonic acid, lactic acid, phosphoric acid, hydrochloric acid, sulphuric acid, tartaric acid and citric acid. Mixtures of the abovementioned acid addition salts may also be used. From the aforementioned acid addition salts the hydrochloride and the hydrobromide, particularly the hydrochloride, are preferred. If flibanserin 1 is used in form of the free base, it is preferably used in form of flibanserin polymorph A as disclosed in WO 03/014079.

The antipsychotic drugs 2 which are suitable to be combined with flibanserin within the teaching of the instant invention and which are mentioned hereinbefore may also be capable of forming acid addition salts with pharmaceutically acceptable acids. Representative salts include the following: Acetate, Benzenesulfonate, Benzoate, Bicarbonate, Bisulfate, Bitartrate, Borate, Bromide, Camsylate, Carbonate, Chloride, Clavulanate, Citrate, Dihydrochoride, Edetate, Edisylate, Estolate, Esylate, Fumarate, Gluceptate, Glyconate, Glutamate, Glycollylarsanilate, Hexylresorcinate, Hydrabamine, Hydrobromide, Hydrochloride, Hydroxynaphthoate, Iodide, Isothionate, Lactate, Lactobionate, Luarate, Malate, Maleate, Mandelate, Mesylate, Methylbromide, Methylnitrate, Methylsulfate, Mucate, Napsylate, Nitrate, N-methylglucamine ammonium salt, Oleate, Oxalate, Pamoate (Embonate), Palmitate, Pantothenate, Phosphate/diphosphate, Polygalacturonate, Salicylate, Stearate, Sulfate, Subacetate, Succinate, Tannate, Tartrate, Teoclate, Tosylate, Triethiodide and Valerate.

Furthermore, where the compounds 2 carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts.

The compounds 2 may have chiral centers and occur as racemates, racemic mixtures and as individual diastermers, or enantiomers with all isomeric forms being included in the present invention. Therefore, where a compound is chiral, the separate enantiomers, substantially free of the other, are included within the scope of the invention. Further included are all mixtures of the two enantiomers. Also included within the scope of the invention are polymorphs and hydrates of the compounds of the instant invention.

The present invention includes within its scope prodrugs of the compounds 1 and 2. In general, such prodrugs will be functional derivatives of the compounds of this invention which are readily convertible in vivo into the required compound.

The term “therapeutically effective amount” shall mean that amount of a drug or pharmaceutical agent that will elicit the biological or medical response of a tissue system animal or human that is being sought by a researcher or clinician.

As used herein, the term “composition” is intended to encompass a product comprising the specified ingredients in the specified amounts, as well as any product which results, directly or indirectly, from combination of the specified ingredients in the specified amounts.

As used herein, the term antipsychotic drug includes all agents that control agitated psychotic behavior, alleviate acute psychotic states, reduce psychotic symptoms, and exert a quieting effect.

In the present invention the term “modulator” means compounds that produce tissue specific effects that can be agonistic or antagonistic.

As used herein, the term “schizophrenia” includes but is not limited to the disorganized type, the catatonic type, the paranoid type, the undifferentiated type, the residual type of schizophrenia, schizoaffective disorder, schizophreniform disorder, delusional disorder, brief psychotic disorder, shared psychotic disorder, psychotic disorder due to a general medical condition, substance-induced psychotic disorder, and psychotic disorder not otherwise specified.

In the combination of the present invention, the components 1 and 2 may be administered separately or together in one pharmaceutical composition. In addition, the administration of one element of the combination of the present invention may be prior to, concurrent to, or subsequent to the administration of the other element of the combination.

The elements of the combination of 1 and 2 may be administered by oral, parenteral (e.g., intramuscular, intraperitoneal, intravenous or subcutaneous injection or implant), buccal, nasal, vaginal, rectal, sublingual, or topical (e.a.. ocular eyedrop) routes of administration and may be formulated, alone or together, in suitable dosage unit formulations containing conventional non-toxic pharmaceutically acceptable carriers, adjuvants and vehicles appropriate for each route of administration.

The pharmaceutical compositions for the administration of the components 1 and 2 of this invention may conveniently be presented in dosage unit form and may be prepared by any of the methods well known in the art of pharmacy. All methods include the step of bringing the active ingredient into association with the carrier which is constituted of one or more accessory ingredients. In general, the pharmaceutical compositions are prepared by uniformily and intimately bringing the active ingredients into association with a liquid carrier or a finely divided solid carrier or both, and then, if necessary, shaping the product into the desired dosage form. In the pharmaceutical compositions the active compounds are included in an amount sufficient to produce the desired pharmacologic effect.

The pharmaceutical compositions containing the active ingredients 1 and 2, separately or together, that are suitable for oral administration may be in the form of discrete units such as hard or soft capsules, tablets, troches or lozenges, each containing a predetermined amount of the active ingredients; in the form of a dispersible powder or granules; in the form of a solution or a suspension in an aqueous liquid or non-aqueous liquid; in the form of syrups or elixirs; or in the form of an oil-in-water emulsion or a water-in-oil emulsion.

Dosage forms intended for oral use may be prepared according to any method known to the art for the manufacture of pharmaceutical formulations and such compositions.

The excipients used may be for example,
(a) inert diluents such as mannitol, sorbitol, calcium carbonate, pregenatinized starch, lactose, calcium phosphate or sodium phosphate;
(b) granulating and disintegrating agents, such as povidone, copovidone, hydroxypropylmethylcellulose, corn starch, alginic acid, crospovidone, sodiumstarchglycolate, croscarmellose, or polacrilin potassium;
(c) binding agents such as microcrystalline cellulose or acacia; and
(d) lubricating agents such as magnesium stearate, stearic acid, fumaric acid or talc.

In some cases, formulations for oral use may be in the form of hardgelatin or HPMC capsules wherein the active ingredient 1 or 2, separately or together, is mixed with an inter solid diluent, for example pregenatinized starch, calcium carbonate, calcium phosphate, or kaolin, or dispensed via a pellet formulation.

They may also be in the form of soft gelatin capsules wherein the active ingredient is mixed with water or an oil medium, for example peanut oil, liquid paraffin, medium chain triglycerides or olive oil.

The tablets, capsules or pellets may be uncoated or they may be coated by known techniques to delay disintegration and absorption in the gastrointestinal tract and thereby provide a delayed action or sustained action over a longer period. For example, a time delay material such as celluloseacetate phtalate or hydroxypropylcellulose acetate succinate or sustained release material such as ethylcellulose or ammoniomethacrylate copolymer (type B) may be employed.

Liquid dosage forms for oral administration include pharmaceutically acceptable emulsions, solutions, suspensions, syrups, and elixirs containing inert diluents commonly used in the art, such as water. Besides such inert diluents, compositions can also include adjuvents, such as wetting agents, emulsifying and suspending agents, and sweetening, flavoring, perfuming and preserving agents.

Aqueous suspensions normally contain the active materials 1 and 2, separately or together, in admixture with excipients suitable for the manufacture of aqueous suspensions. Such excipients may be
(a) suspending agents such as hydroxy ethylcellulose, sodium carboxymethylcellulose, methylcellulose, hydroxypropylmethylcellulose, sodium alginate, polyvinylpyrrolidone, gum tragacanth and gum acacia;
(b) dispersing or wetting agents which may be

(b.1) a naturally-occuring phosphatide such as lecithin,

(b.2) a condensation product of an alkylene oxide with a fatt acid, for example, polyoxyethylene stearate,

(b.3) a condensation product of ethylene oxide with a long chain aliphatic alcohol, for example heptadecaethyleneoxycetanol,

(b.4) a condensation product of ethylene oxide with a partial ester derived from a fatty acid and a hexitol such as polyoxyethylene sorbitol monooleate, or

(b.5) a condensation product of ethylene oxide with a partial ester derived from a fatty acide and a hexitol anhyrdride, for example polyoxyethylene sorbitan monooleate.

The aqueous suspensions may also contain one or more preservatives, for example, ethyl or n-propyl p-hydroxybenzoate; one or more coloring agents; one or more flavoring agents; and one or more sweetening agents, such as sucrose or saccharin.

Oily suspensions may be formulated by suspending the active ingredients 1 and 2, separately or together, in a vegtable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents and flavoring agents may be added to provide a palatable oral preparation. These compositions may be prepared by the addition of an antioxidant such as ascorbic acid.

Dispersible powders and granules are suitable for the preparation of an aqueous suspension. They provide the active ingredients 1 and 2, separately or together, in admixture with a dispersing or wetting agent, a suspending agent and one or more preservatives. Suitable dispersing or wetting agents and suspending agents are exemplified by those already mentioned above. Additional excipients, for example, those sweetening, flavoring and coloring agents described above may also be present.

The pharmaceutical compositions of the invention may also be in the form of oil-in-water emulsions. The oily phase may be a vegtable oil such as olive oil or arachis oils, or a mineral oil such as liquid paraffin or a mixture thereof.

Suitable emulsifying agents may be
(a) naturally-occuring gums such as gum acacia and gum tragacanth,
(b) naturally-occuring phosphatides such as soybean and lecithin,
(c) esters or partial esters derived from fatty acids and hexitol anhydrides, for example, sorbitan monooleate,
(d) condensation products of said partial esters with ethylene oxide, for example polyoxyethylene sorbitan monooleate. The emulsions may also contain sweetening and flavoring agents.

Syrups and elixirs may be formulated with sweetening agents, for example, glycerol, propylene glycol, sorbitol or sucrose. Such formulations may also contain a preservative and flavoring and coloring agents.

The pharmaceutical compositions containing 1 and 2, separately or together, may be in the form of a sterile injectable aqueous or oleagenous suspension or solution. The suspension may be formulated according to known methods using those suitable dispersing or wetting agents and suspending agents which have been mentioned above. The sterile injectable preparation may also be a sterile injectable solution or suspension in a non toxic parenterally-acceptable diluent or solvent, for example as a solution in 1,3-butane-diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono-or diglycerides. In addition, fatty acids such as oleic acid find use in the preparation of injectables.

Preparations according to this invention containing 1 and 2, separately or together, for parenteral administration include sterile aqueous or non-aqueous solutions, suspension, or emulsions.

Examples of non-aqueous solvents or vehicles are propylene glycol, polyethylene glycol, vegetable oils, such as olive oil and corn oil, gelatin, and injectable organic esters such as ethyl oleate. Such dosage forms may also contain adjuvants such as preserving, wetting, emulsifying, and dispersing agents. They may be sterilized by, for example, filtration through a bacteria-retaining filter, by incorporating sterilizing agents into the compositions, by irradiating the compositions, or by heating the compositions. They can also be manufactured in the form of sterile solid compositions which can be reconstitured in sterile water, or some other sterile injectable medium immediately before use. The combination of this invention may also be administered in the form of suppositories for rectal administration. This composition can be prepared by mixing the drugs with a suitable non-irritating exipient which is solidat ordinary temperatures but liquid at the rectal temperature and will therefore melt in the rectum to release the drug. Such materials are cocoa butter, hard fat, and polyethylene glycols. Compositions for buccal, nasal or sublingual administration are also prepared with standard excipients well known in the art.

For topical administration the combinations of this invention containing 1 and 2, separately or together, may be formulated in liquid or semi-liquid preparations such as miniments, lotions, applications; oil-in-water or water-in-oil emulsions such as creams, ointments, jellies or pastes, including tooth-pastes; or solutions or suspensions such as drops, and the like.

The dosage of the active ingredients in the compositions of this invention may be varied. However, it is necessary that the amount of the active ingredients 1 and 2 be such that a suitable dosage form is obtained. The selected dosage and the dosage form depend upon the desired therapeutic effect, on the route of administration and on the duration of the treatment. Dosage ranges in the combination are approximately one tenth to one times the clinically effective ranges required to induce the desired therapeutic effect, respecitvely when the compounds are used singly.

Within the instant invention flibanserin 1 is preferably administered in such an amount that per single dosage between 5 to 200 mg of flibanserin 1 are applied. Preferred are ranges of between 10 to 150 mg, particular preferred 20 to 100 mg of flibanserin 1. Suitable dosage forms may contain for instance 20, 25, 30, 35, 40, 45, 50, 55, 60, 65, 70, 75, 80, 85, 90, 95 or 100 mg of flibanserin 1. The aforementioned values are based on flibanserin 1 in form of the free base. If flibanserin 1 is applied in form of one of its acid addition salts, the corresponding values are readily calculable from the aforementioned values.

Within the instant invention the additional antipsychotic drug 2 is preferably administered in such an amount that per day between 0,1 to 2,500 mg of 2 are applied. Preferred are ranges of between 0,5 to 2,000 mg, in particular between 1 to 1,000mg.

Suitable dosage forms may contain for instance 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2, 2.05, 2.1, 2.15, 2.2, 2.25, 2.3, 2.35, 2.4, 2.45, 2.5, 2.55, 2.6, 2.65, 2.7, 2.75, 2.8, 2.85, 2.9, 2.95, 3, 3.05, 3.1, 3.15, 3.2, 3.25, 3.3, 3.45, 3.4, 3.45, 3.5, 3.55, 3.6, 3.65, 3.7, 3.75, 3.8, 3.85, 3.9, 3.95, 4, 4.1, 4.15, 4.2, 4.25, 4.3, 4.35, 4.4, 4.45, 4.5, 4.55, 4.6, 4.65, 4.7, 4.75, 4.8, 4.85, 4.9, 4.95, 5, 10, 15, 20, 25, 30, 35, 40, 45, 50, 55, 60, 65, 70, 76, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, or 450, 475, 500, 520, 550, 575, 600, 625, 650, 675, 700, 725, 750, 800, 825, 850, 875, 900, 925, 950, 975, or 1,000 mg or 2. Advantageously, the compounds 2 of the present invention may be administered in a single daily dose, or the total daily dosage may be administered in divided doses of two, three or four times daily.

In another preferred embodiment the invention related to a method for the treatment and/or prevention of schizophrenia, comprising the administration of a therapeutically effective amount of 1 optionally in form of the free base, the pharmacologically acceptable acid addition salts and/or optionally in form of the hydrates and/or solvates thereof, in combination with a therapeutically effective amount of 2, optionally in form of the pharmaceutically acceptable acid addition salts, in form of the hydrates and/or solvates and optionally in the form of the individual optical isomers, mixtures of the individual enantiomers or racemates thereof, separeately or together within one pharmaceutical composition.

In another preferred embodiment the invention relates to a method for the treatment and/or prevention of schizophrenia and related disorders selected from the group consisting of the disorganized type, the catatonic type, the paranoid type, the undifferentiated type, the residual type of schizophrenia, schizoaffective disorder, schizophreniform disorder, delusional disorder, brief psychotic disorder, shared psychotic disorder, psychotic disorder due to a general medical condition, substance-induced psychotic disorder, and psychotic disorder not otherwise specified, comprising the administration of a therapeutically effective amount of 1 optionally in form of the free base, the pharmacologically acceptable acid addition salts and/or optionally in form of the hydrates and/or solvates thereof, in combination with a therapeutically effective amount of 2, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

In another preferred embodiment the invention relates to a method for the treatment and/or prevention of the disorganied type of schizophrenia, comprising the administration of a therapeutically effective amount of 1 optionally in form of the free base, the pharmacologically acceptable acid addition salts and/or optionally in form of the hydrates and/or solvates thereof, in combination with a therapeutically effective amount of 2, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

In another preferred embodiment the invention relates to a method for the treatment and/or prevention of the catatonic type of schizophrenia, comprising the administration of a therapeutically effective amount of 1 optionally in form of the free base, the pharmacologically acceptable acid addition salts and/or optionally in form of the hydrates and/or solvates thereof, in combination with a therapeutically effective amount of 2, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutial composition.

In another preferred embodiment the invention relates to a method for the treatment and/or prevention of the paranoid type of schizophrenia, comprising the administration of a therapeutically effective amount of 1 optionally in form of the free base, the pharmacologically acceptable acid addition salts and/or optionally in form of the hydrates and/or solvates thereof, in combination with a therapeutically effective amount of 2, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

In another preferred embodiment the invention relates to a method for the treatment and/or prevention of the undifferentiated type of schizophrenia, comprising the administration of a therapeutically effective amount of 1 optionally in form of the free base, the pharmacologically acceptable acid addition salts and/or optionally in form of the hydrates and/or solvates thereof, in combination with a therapeutically effective amount of 2, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

In another preferred embodiment the invention relates to a method for the treatment and/or prevention of the residual type of schizophrenia, comprising the administration of a therapeutically effective amount of 1 optionally in form of the free base, the pharmacologically acceptable acid addition salts and/or optionally in form of the hydrates and/or solvates thereof, in combination with a therapeutically effective amount of 2, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

In another preferred embodiment the invention relates to a method for the treatment and/or prevention of schizoaffective disorder, comprising the administration of a therapeutically effective amount of 1 optionally in form of the free base, the pharmacologically acceptable acid addition salts and/or optionally in form of the hydrates and/or solvates thereof, in combination with a therapeutically effective amount of 2, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

In another preferred embodiment the invention relates to a method for the treatment and/or prevention of schizophreniform disorder, comprising the administration of a therapeutically effective amount of 1 optionally in form of the free base, the pharmacologically acceptable acid addition salts and/or optionally in form of the hydrates and/or solvates thereof, in combination with a therapeutically effective amount of 2, optionally in form of the pharmaceutically acceptable acid addition salts, in form of the hydrates and/or solvates and optionally in the form of the individual optical isomers, mixtures of the individual enentiomers or racemates thereof, separately or together within one pharmaceutical composition.

In another preferred embodiment the invention relates to a method for the treatment and/or prevention of delusional disorder, comprising the administration of a therapeutically effective amount of 1 optionally in form of the free base, the pharmacologically acceptable acid addition salts and/or optionally in form of the hydrates and/or solvates thereof, in combination with a therapeutically effective amount of 2, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

In another preferred embodiment the invention relates to a method for the treatment and/or prevention of brief psychotic disorder, comprising the administration of a therapeutically effective amount of 1 optionally in form of the free base, the pharmacologically acceptable acid addition salts and/or optionally in form of the hydrates and/or solvates thereof, in combination with a therapeutically effective amount of 2, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

In another preferred embodiment the invention relates to a method for the treatment and/or prevention of shared psychotic disorder, comprising the administration of a therapeutically effective amount of 1 optionally in form of the free base, the pharmacologically acceptable acid addition salts and/or optionally in form of the hydrates and/or solvates thereof, in combination with a therapeutically effective amount of 2, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

In another preferred embodiment the invention relates to a method for the treatment and/or prevention of psychotic disorder due to a general medical condition, comprising the administration of a therapeutically effective amount of 1 optionally in form of the free base, the pharmacologically acceptable acid addition salts and/or optionally in form of the hydrates and/or solvates thereof, in combination with a therapeutically effective amount of 2, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

In another preferred embodiment the invention relates to a method for the treatment and/or prevention of the substance-induced psychotic disorder, comprising the administration of a therapeutically effective amount of 1 optionally in form of the free base, the pharmacologically acceptable acid addition salts and/or optionally in form of the hydrates and/or solvates thereof, in combination with a therapeutically effective amount of 2, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

In another preferred embodiment the invention relates to a method for the treatment and/or prevention of psychotic disorder not otherwise specified, comprising the administration of a therapeutically effective amount of 1 optionally in form of the free base, the pharmacologically acceptable acid addition salts and/or optionally in form of the hydrates and/or solvates thereof, in combination with a therapeutically effective amount of 2, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

The beneficial effects of the compositions according to the invention can be observed regardless of whether the disturbance existed lifelong or was acquired, and independent of etiologic origin (organic—both, physically and drug induced—, psychogen, a combination of organic—both, physically and drug induced—, and psychogen, or unknown).

Another embodiment of the invention is directed to the aforementioned methods wherein 2 is selected from the group consisting of 5HT_{1A} agnostics, dopamine modulators, sodium channel blockers, 5-HT uptake inhibitors, D3 antagonists, D2 antagonists, D1 antagonists, D1 agonists, secretin agonist, phospholipase A2 inhibitors, 5-HT2 antagonists, 5HT6 antagonists, COX 2 inhibitors, 5-HT_{2A} antagonists, 5HT_{2C} modulators, NK3 antagonists, alpha 1 adrenoreceptor antagonists, alpha 2 adrenoreceptor antagonists, AMPA modulators and NK 3 antagonists.

Another embodiment of the invention is directed to the aforementioned methods wherein 2 is selected from the group consisting of D2 antagonists.

Another preferred embodiment of the invention is directed to the aforementioned methods wherein 2 is selected from the group consisting of include Chlorpromazine, Thioridazine, Haloperidol, Perphenazine, Thiothixene, Trifluoperazine, Fluphenazine, Clozapine, Risperidone, Olanzapine, Quetiapine, Pimozide, Aripiprazole, Ziprasidone, Perospirone, Nemonapride, Sertindole, Levosulpiride, Tandospirone, Bifeprunox, Asenapine, Paliperidone, Mifepristone, Lamotrigine, Iloperidone, Blonanserin, DU-125530, Lurasidone, ACP-103, Idazoxan, Org-24448, CX-516, Aplindore, SLV-313, SLV-310, Ocaperidone, PNU-170413, POL-255, ABT-089 Talnetant, NE-100, LAX-101, LAX-111, RG-1068 (Secretin), Dexefraoxan, Dihydrexidine, SM-13496, D-Serine, Osanetant, EMR-62218, SB-399885, TC-1698, SR-147778, SLV-319, SSR-181507, AVE-5997, PNU-177864, Abaperidone, SSR-146977, Neboglamine, Lamictal XT, N-Desmethylclozapine, Topiramate and cycloserine, optionally in form of the pharmaceutically acceptable acid addition salts, in form of the hydrates and/or solvates and optionally in the form of the individual optical isomers, mixtures of the individual enantiomers or racemates thereof.

Another embodiment of the invention relates to the use of the combinations of 1, optionally in form the free base, the pharmacologically acceptable acid addition salts and/or optionally in form of the hydrates and/or solvates thereof, and one or more additional antipsychotic drugs 2, optionally in form of the pharmaceutically acceptable salts, in form of the hydrates and/or solvates and optionally in the form of the individual optical isomers, mixtures of the individual enantiomers or racemates thereof, for the preparation of a medicament for the treatment and/or prevention of the aforementioned disorders.

Another embodiment of the invention relates to the use of the combinations of 1, optionally in form the free base, the pharmacologically acceptable acid addition salts and/or optionally in form of the hydrates and/or solvates thereof, and 2, optionally in form of their pharmaceutically acceptable acid addition salts for the preparation of a medicament for the treatment and/or prevention of the aforementioned disorders, wherein 260 is selected from the group consisting of 5HT_{1A} agnostics, dopamine modulators, sodium channel blockers, 5-HT uptake inhibitors, D3 antagonists, D2 antagonists, D1 antagonists, D1 agonists, secretin agonist, phospholipase A2 inhibitors, 5-HT2 antagonists, 5HT6 antagonists, COX 2 inhibitors, 5-HT_{2A} antagonists, 5HT_{2C} modulators, NK3 antagonists, alpha 1 adrenoreceptor antagonists, alpha 2 adrenoreceptor antagonists, AMPA modulators and NK 3 antagonists.

Another embodiment of the invention relates to the use of the combinations of 1, optionally in form of the free base, the pharmacologically acceptable acid addition salts and/or optionally in form of the hydrates and/or solvates thereof, and 2, optionally in form of their pharmaceutically acceptable acid addition salts for the preparation of a medicament for the treatment and/or prevention of the aforementioned disorders, wherein 2 is selected from the group consisting of Clorpromazine, Thioridazine, Haloperidol, Perphenazine, Thiothixene, Trifluoperazine, Fluphenazine, Clozapine, Risperidone, Olanzapine, Quetiapine, Pimozide, Aripiprazole, Ziprasidone, Perospirone, Nemonapride, Sertindole, Levosulpiride, Tandospirone, Bifeprunox, Asenapine, Paliperidone, Mifepristone, Lamotrigine, Iloperidone, Blonanserin, DU-125530, Lurasidone, ACP-103, Idazoxan, Org-24448, CX-516, Aplindore, SLV-313, SLV-310, Ocaperidone, PNU-170413, POL-255, ABT-089 Talnetant, NE-100, LAX-101, LAX-111, RG-1068 (Secretin), Dexefaroxan, Dihydrexidine, SM-13496, D-Serine, Osanetant, EMR-62218, SB-399885, TC-1698, SR-1447778, SLV-319, SSR-181507, AVE-5997, PNU-177864, Abaperidone, SSR-146977, Neboglamine, Lamictal XR, N-Desmethylclozapine, Topiramate and cycloserine, optionally in form of the pharmaceutically acceptable acid addition salts, in form of the hydrates and/or solvates and optionally in the form of the individual optical isomers, mixtures of the individual enantiomers or racemates thereof.

The following examples demonstrate possible pharmaceutical compositions comprising flibanserin in combination with one of the aforementioned combination partners 2.

EXAMPLE N^{o}1 - Combination 1 with chlorpromazine

| | |
|---|---|
| Constituents | mg/tablet |

Core

| | |
|---|---|
| Flibanserin (free base) | 50.000 |
| Chlorpromazine hydrochloride | 20.000 |
| Anhydrous dibasic calcium phosphate | 100.000 |
| Microcrystalline cellulose | 203.090 |
| HPMC (Methocel E5) | 6.615 |
| Croscarmellose sodium | 8.820 |
| Magnesium stearate | 2.250 |

Coating

| | |
|---|---|
| HPMC (Methocel E5) | 4.320 |
| Polyethylene Glycol 6000 | 1.260 |
| Titanium dioxide | 1.800 |
| Talc | 1.542 |
| Iron oxide red | 0.078 |
| Total Film coated tablet | 399.775 |

EXAMPLE N^{o}2 - Combination 1 with clozapine

| | |
|---|---|
| Constituents | mg/tablet |

Core

| | |
|---|---|
| Flibanserin (free base) | 50.000 |
| Clozapine | 100.000 |
| Lactose monohydrate | 133.750 |
| Microcrystalline cellulose | 40.000 |
| Hydroxypropylcellulose | 2.500 |
| Corn starch | 12.500 |
| Magnesium stearate | 1.250 |

Coating

| | |
|---|---|
| HPMC (e.g. Pharmacoat 606) | 2.400 |
| Polyethylene Glycol 6000 | 0.700 |
| Titanium dioxide | 1.000 |
| Talc | 0.857 |
| Iron oxide yellow | 0.043 |
| Total Film coated tablet | 345.000 |

EXAMPLE N^{o}3 - Combination 1 with alprazolam

| | |
|---|---|
| Constituents | mg/tablet |

Core

| | |
|---|---|
| Flibanserin (free base) | 50.000 |
| Fluphenazine hydrochloride | 5.000 |
| Lactose monohydrate | 143.490 |
| Microcrystalline cellulose | 47.810 |
| HPMC (e.g. Pharmacoat 606) | 2.500 |
| Carboxymethylcellulose sodium | 5.000 |
| Mannitol | 60.000 |
| Corn starch | 36.500 |
| Povidone | 1.000 |
| Colloidal silicon dioxide | 1.000 |
| Magnesium stearate | 1.700 |

Coating

| | |
|---|---|
| HPMC (e.g. Methocel E5) | 3.360 |
| Polyethylene Glycol 6000 | 0.980 |
| Titanium dioxide | 1.400 |
| Talc | 1.200 |
| Iron oxide red | 0.060 |
| Total Film coated bilayer tablet | 362.000 |

EXAMPLE N^{o}4 - Combination of 1 with citalopram

| | |
|---|---|
| Constituents | mg/tablet |

Final Mixture

| | |
|---|---|
| Flibanserin (free base) | 50.000 |
| Haloperidol | 20.000 |
| Lactose monohydrate | 200.000 |
| Pregelatinized starch | 108.000 |
| Magnesium stearate | 2.000 |

Capsule

| | |
|---|---|
| Final Mixture | 380.000 |
| Capsule (size 1) | 82.000 |
| Total weight of Capsule | 462.000 |

The following examples show preferred pharmaceutical compositions of flibanserin, if the combinations according to the invention are administered in separate dosage units.

EXAMPLE N^{o}5 - Composition

| | |
|---|---|
| Constituents | mg/tablet |

Core

| | |
|---|---|
| Flibanserin (free base) | 25.000 |
| Lactose monohydrate | 71.720 |
| Microcrystalline cellulose | 23.905 |
| HPMC (Methocel E5) | 1.250 |
| Carboxymethylcellulose sodium | 2.500 |
| Magnesium stearate | 0.625 |

Coating

| | |
|---|---|
| HPMC (Methocel E5) | 1.440 |
| Polyethylene Glycol 6000 | 0.420 |
| Titanium dioxide | 0.600 |
| Talc | 0.514 |
| Iron oxide red | 0.026 |
| Total Film coated tablet | 128.000 |

EXAMPLE N^{o}6 - Composition

| | |
|---|---|
| Constituents | mg/tablet |

Core

| | |
|---|---|
| Flibanserin (free base) | 50.000 |
| Lactose monohydrate | 143.440 |
| Microcrystalline cellulose | 47.810 |
| HPMC (e.g. Pharmacoat 606) | 2.500 |
| Carboxymethylcellulose sodium | 5.000 |
| Magnesium stearate | 1.250 |

Coating

| | |
|---|---|
| HPMC (e.g. Pharmacoat 606) | 2.400 |
| Polyethylene Glycol 6000 | 0.700 |
| Titanium dioxide | 1.000 |
| Talc | 0.857 |
| Iron oxide red | 0.043 |
| Total Film coated tablet | 255.000 |

Example N^{o}7 - Composition

| | |
|---|---|
| Constituents | mg/tablet |

Core

| | |
|---|---|
| Flibanserin (free base) | 100.000 |
| Lactose monohydrate | 171.080 |
| Microcrystalline cellulose | 57.020 |
| HPMC (e.g. Methocel E5) | 3.400 |
| Carboxymethylcellulose sodium | 6.800 |
| Magnesium stearate | 1.700 |

Coating

| | |
|---|---|
| HPMC (e.g. Methocel E5) | 3.360 |
| Polyethylene Glycol 6000 | 0.980 |
| Titanium dioxide | 1.400 |
| Talc | 1.200 |
| Iron oxide red | 0.060 |
| Total Film coated tablet | 347.000 |

EXAMPLE N^{o}8 - Composition

| | |
|---|---|
| Constituents | mg/tablet |

Core

| | |
|---|---|
| Flibanserin (free base) | 2.000 |
| Dibasic Calciumphosphate, anhydrous | 61.010 |
| Microcrystalline cellulose | 61.010 |
| HPMC (Methocel E5) | 1.950 |
| Carboxymethylcellulose sodium | 2.600 |
| Colloidal silicon dioxide | 0.650 |
| Magnesium stearate | 0.780 |

Coating

| | |
|---|---|
| HPMC (Methocel E5) | 1.440 |
| Polyethylene Glycol 6000 | 0.420 |
| Titanium dioxide | 0.600 |
| Talc | 0.514 |
| Iron oxide red | 0.026 |
| Total Film coated tablet | 133.000 |

EXAMPLE N^{o}9 - Composition

| | |
|---|---|
| Constituents | mg/tablet |

Core

| | |
|---|---|
| Flibanserin (free base) | 100.000 |
| Dibasic Calciumphosphate, anhydrous | 69.750 |
| Microcrystalline cellulose | 69.750 |
| HPMC (e.g. Methocel E5) | 2.750 |
| Carboxymethylcellulose sodium | 5.000 |
| Colloidal silicon dioxide | 1.250 |
| Magnesium stearate | 1.500 |

Coating

| | |
|---|---|
| HPMC (e.g. Methocel E5) | 2.400 |
| Polyethylene Glycol 6000 | 0.700 |
| Titanium dioxide | 1.043 |
| Talc | 0.857 |
| Total Film coated tablet | 255.000 |

EXAMPLE N^{o}10 - Composition

| | |
|---|---|
| Constituents | mg/tablet |

Core

| | |
|---|---|
| Flibanserin (free base) | 20.000 |
| Lactose monohydrate | 130.000 |
| Microcrystalline cellulose | 43.100 |
| Hydroxypropyl Cellulose (e.g. Klucel LF) | 1.900 |
| Sodium Starch Glycolate | 4.000 |
| Magnesium stearate | 1.000 |

Coating

| | |
|---|---|
| HPMC (e.g. Methocel E5) | 2.400 |
| Polyethylene Glycol 6000 | 0.700 |
| Titanium dioxide | 1.043 |
| Talc | 0.857 |
| Total Film coated tablet | 205.000 |

What is claimed is:

1. A pharmaceutical composition comprising a therapeutically effective amount of flibanserin, in the form of a free base or a pharmacologically acceptable acid addition salt, in combination with a therapeutically effective amount of an additional antipsychotic drug.

2. The pharmaceutical composition according to claim 1, wherein the additional antipsychotic drug is selected from the group consisting of 5HT_{1A} agnostics, dopamine modulators, sodium channel blockers, 5-HT uptake inhibitors, D3 antagonists, D2 antagonists, D1 antagonists, D1 agonists, secretin agonist, phospholipase A2 inhibitors, 5-HT2 antagonists, 5HT6 antagonists, COX 2 inhibitors, 5-HT_{2A} antagonists, 5HT_{2C} modulators, NK3 antagonists, alpha 1 adrenoreceptor antagonists, alpha 2 adrenoreceptor antagonists, AMPA modulators and NK 3 antagonists.

3. The pharmaceutical composition according to claim 1, wherein the additional antipsychotic drug is a D2 antagonist.

4. The pharmaceutical composition according to claim 1, wherein the additional antipsychotic drug is selected from the group consisting of Chlorpromazine. Thioridazine, Haloperidol, Perphenazine, Thiothixene, Trifluoperazine, Fluphenazine, Clozapine, Risperidone, Olanzapine, Quetiapine, Pimozide, Aripiprazole, Ziprasidone, Perospirone, Nemonapride, Sertindole, Levosulpiride, Tandospirone, Bifeprunox, Asenapine, Paliperidone, Mifepristone, Lamotrigine, Iloperidone, Blonanserin, DU-125530, Lurasidone, ACP-103, Idazoxan, Org-24448, CX-516, Aplindore, SLV-313, SLV-310, Ocaperidone, PNU-170413, POL-255, ABT-089 Talnetant, NE-100, LAX-101, LAX-111, RG-1068 (Secretin), Dexefraoxan, Dihydrexidine, SM-13496, D-Serine, Osanetant, EMR-62218, SB-399885, TC-1698, SR-147778, SLV-319, SSR-181507, AVE-5997, PNU-177864, Abaperidone, SSR-146977, Neboglamine, Lamictal XT, N-Desmethylclozapine, Topiramate and cycloserine.

5. The pharmaceutical composition according to claim 1, wherein flibanerin, in the form of a free base or a pharmacologically acceptable acid addition salt, and the additional antipsychotic drug are together in one dosage form.

6. The pharmaceutical composition according to claim 1, wherein flibanerin, in the form of a free base or a pharmacologically acceptable acid addition salt, and the additional antipsychotic drug are separate in one dosage form.

7. The pharmaceutical composition of claim 1, wherein flibanserin, in the form of a free base or a pharmacologically acceptable acid addition salt, is a hydrate and/or a solvate.

8. The pharmaceutical composition of claim 1, wherein the additional antipsychotic drug is in the form of a pharmaceutically acceptable acid addition salt.

9. The pharmaceutical composition of claim 1, wherein the additional antipsychotic drug is a hydrate and/or a solvate.

10. The pharmaceutical composition of claim 1, wherein the additional antipsychotic drug is an individual optical isomer, a mixture of individual enantiomers or racemates thereof.

11. A method for the treatment and/or prevention of schizophrenia and related diseases, comprising the administration of a therapeutically effective amount of flibanserin, in the form of a free base or a pharmacologically acceptable acid addition salt, in combination with a therapeutically effective amount of an additional antipsychotic drug.

12. A method according to claim 11 wherein the schizophrenia and related diseases are selected from the group consisting of the disorganized type of the catatonic type of schizophrenia, the paranoid type of schizophrenia, the undifferentiated type of schizophrenia, the residual type of schizophrenia, schizoaffective disorder, schizophreniform disorder, delusional disorder, brief psychotic disorder, shared psychotic disorder, psychotic disorder due to a general medical condition, substance-induced psychotic disorder, and other psychotic disorders.

13. A method according to claim 11 or 12, wherein the additional antipsychotic drug is selected from the group consisting of 5HT_{1A} agnostics, dopamine modulators, sodium channel blockers, 5-HT uptake inhibitors, D3 antagonists, D2 antagonists, D1 antagonists, D1 agonists, secretin agonist, phospholipase A2 inhibitors, 5-HT2 antagonists, 5HT6 antagonists, COX 2 inhibitors, 5-HT_{2A} antagonists, 5HT_{2C} modulators, NK3 antagonists, alpha 1 adrenoreceptor antagonists, alpha 2 adrenoreceptor antagonists, AMPA modulators and NK 3 antagonists.

14. A method according to claim 11 or 12, wherein the additional antipsychotic drug is a D2 antagonist.

15. A method according to claim 11 or 12, wherein the additional antipsychotic drug is selected from the group consisting of Chlorpromazine, Thioridazine, Haloperidol, Perphenazine, Thiothixene, Trifluoperazine, Fluphenazine, Clozapine, Risperidone, Olanzapine, Quetiapine, Pimozide, Aripiprazole, Ziprasidone, Perospirone, Nemonapride, Sertindole, Levosulpiride, Tandospirone, Bifeprunox, Asenapine, Paliperidone, Mifepristone, Lamotrigine, Iloperidone, Blonanserin, DU-125530, Lurasidone, ACP-103, Idazoxan, Org-24448, CX-516, Aplindore, SLV-313, SLV-310, Ocaperidone, PNU-170413, POL-255, ABT-089 Talnetant, NE-100, LAX-101, LAX-111, RG-1068 (Secretin), Dexefraoxan, Dihydrexidine, SM-13496, D-Serine, Osanetant, EMR-62218, SB-399885, TC-1698, SR-147778, SLV-319, SSR-181507, AVE-5997, PNU-177864, Abaperidone, SSR-146977, Neboglamine, Lamictal XT, N-Desmethylclozapine, Topiramate and cycloserine.

16. The method of claim 11, wherein flibanserin, in the form of a free base or a pharmacologically acceptable acid addition salt, and the additional antipsychotic drug are administered separately, each in one dosage form.

17. The method of claim 11, wherein flibanserin, in the form of a free base or a pharmacologically acceptable acid addition salt, and the additional antipsychotic drug are administered together within one dosage form.
